(19) United States
(12) Patent Application Publication
(10) Pub. No.: US 2026/0132046 A1
(43) Pub. Date: May 14, 2026

(54) CHIRAL MANGANESE DIOXIDE NANOPARTICLE AND PREPARATION METHOD THEREFOR AND APPLICATION THEREOF

(71) Applicant: JIANGNAN UNIVERSITY, Wuxi (CN)

(72) Inventors: Chuanlai XU, Wuxi (CN); Aihua QU, Wuxi (CN); Hua KUANG, Wuxi (CN); Liguang XU, Wuxi (CN); Maozhong SUN, Wuxi (CN); Xiaoling WU, Wuxi (CN); Liqiang LIU, Wuxi (CN); Changlong HAO, Wuxi (CN); Shanshan SONG, Wuxi (CN); Yongming HU, Wuxi (CN); Aihong WU, Wuxi (CN); Lingling GUO, Wuxi (CN); Xinxin XU, Wuxi (CN)

(21) Appl. No.: 19/386,065

(22) Filed: Nov. 11, 2025

Related U.S. Application Data
(63) Continuation of application No. PCT/CN2024/087165, filed on Apr. 11, 2024.

(30) Foreign Application Priority Data
Sep. 22, 2023 (CN) 202311232862.2

(51) Int. Cl. C01G 45/024; A61K 8/19; A61Q 19/08

(52) U.S. Cl. CPC C01G 45/024; A61K 8/19; A61Q 19/08; A61K 2800/413; C01P 2004/04; C01P 2004/64

(57) ABSTRACT

The invention provides a chiral manganese dioxide nanoparticle and a preparation method and application thereof. The chiral manganese dioxide nanoparticle having good biocompatibility is prepared by taking D-gluconic acid as a ligand. The chiral manganese dioxide nanoparticle of the present invention has a strong circular dichroism signal in a visible light region, so that interference of signals in a small molecule ultraviolet region in an organism can be avoided; and by means of the redox reaction with ROS, change in the circular dichroism signal intensity of the chiral manganese dioxide nanoparticle presents a good linear relationship with the concentration of ROS, so that the quantitative detection of ROS is achieved. The chiral manganese dioxide nanoparticle prepared in the present invention can effectively remove ROS in aged skin, increase dermis thickness and collagen proportion, and reduce the oxidative stress level in skin tissue, and is beneficial to delaying skin aging.

### FIELD OF THE INVENTION

The present disclosure relates to the technical field of material chemistry, and in particular to a chiral manganese dioxide nanoparticle, a preparation method therefor, and use thereof.

### DESCRIPTION OF THE RELATED ART

ROS are products of aerobic metabolism in the body and include superoxide, hydroxyl radical, hydrogen peroxide, hypochlorous acid, and the like. Maintaining the dynamic balance of ROS plays an important role in intracellular signal transduction and regulation, gene expression, and organismal homeostasis. It has been reported that oxidative damage caused by excessive ROS accumulation can lead to DNA damage, lipid peroxidation, and disruption of protein structure and function, resulting in aging and aging-related diseases. Aging is manifested most intuitively on the skin, characterized by visible signs such as skin loosening, increased wrinkles, reduced collagen, and thinning of the epidermis and dermis. In addition, skin aging is also accompanied by a reduction in barrier and defense functions, resulting in an increased incidence of related diseases.

Traditional ROS detection and analysis methods include chemiluminescence, fluorescent probes, and electron paramagnetic resonance. However, these methods have limited applications due to poor biocompatibility, low sensitivity, and interference from the autofluorescence of biological substrates. In recent years, with the development and progress of nanotechnology, chiral nanomaterials have been widely applied in the fields of biodetection, bioimaging, disease diagnosis and treatment, and the like. Therefore, the preparation of a novel chiral manganese dioxide nanoparticle, which can quantitatively detect ROS by utilizing the reversible redox reaction between manganese particles and ROS and effectively eliminate excessive ROS accumulation in aged skin, has important research significance for alleviating age-related diseases, particularly for combating skin aging.

### SUMMARY OF THE INVENTION

To address the defects of the existing detection technologies, the present disclosure provides a chiral manganese dioxide nanoparticle, a preparation method therefor, and use thereof, namely a method for preparing chiral manganese dioxide with good biocompatibility, and the method realizes real-time quantitative detection of ROS in living cells by utilizing changes in circular dichroism signals in the visible light region, so that interference from circular dichroism signals of intracellular molecules in the ultraviolet region is successfully avoided. Meanwhile, a reversible redox reaction occurs between the chiral manganese dioxide nanoparticle and ROS, so that excessive ROS accumulation in aged skin can be effectively eliminated, thereby alleviating skin aging.

The present disclosure is implemented by the following technical solutions:
A first object of the present disclosure is to provide a method for preparing a chiral manganese dioxide nanoparticle, which comprises the following steps:
mixing a manganese source with chiral gluconic acid in a solvent, and adjusting the pH to alkalinity to give the chiral manganese dioxide nanoparticle.

In one embodiment of the present disclosure, one or more of the following conditions are met:
(1) the chiral gluconic acid is D-gluconic acid or L-gluconic acid;
(2) the manganese source is selected from manganese chloride and/or manganese nitrate;
(3) the solvent is selected from the group consisting of ethylene glycol, ethanol, polyethylene glycol, and any combination thereof.
A second object of the present disclosure is to provide the chiral manganese dioxide nanoparticle prepared by the preparation method.

In one embodiment of the present disclosure, one or two of the following conditions are met:
(a) the chiral manganese dioxide nanoparticle has a particle size of 3 nm-20 nm, preferably 3 nm-6 nm;
(b) the chiral manganese dioxide particle has a circular dichroism spectral signal at 400 nm-700 nm.

In one embodiment of the present disclosure, the pH adjuster is sodium hydroxide.
A third object of the present disclosure is to provide use of the chiral manganese dioxide nanoparticle in the detection of ROS.

In one embodiment of the present disclosure, the chiral manganese dioxide nanoparticle for ROS detection has a circular dichroism spectral signal at 553 nm.

In one embodiment of the present disclosure, the ROS is at a concentration of 0.01 μM-100 μM, preferably 0.01 μM-10 μM.

In one embodiment of the present disclosure, the mechanism of the reaction of the chiral manganese dioxide nanoparticle with ROS is the reduction of tetravalent manganese into divalent manganese and oxygen, and the divalent manganese is further oxidized into tetravalent manganese and water.
A fourth object of the present disclosure is to provide a composition comprising the chiral manganese dioxide nanoparticle.
A fifth object of the present disclosure is to provide use of the chiral manganese dioxide nanoparticle or the composition in the preparation of an anti-aging skincare product, thereby effectively eliminating excessive ROS accumulation in aged skin.

In one embodiment of the present disclosure, the skincare product is a lotion, a cream, a facial mask, an essence, a skincare toner, a facial cleanser, or a gel.

A sixth object of the present disclosure is to provide use of the chiral manganese dioxide nanoparticle or the composition in the preparation of an anti-aging medicament or healthcare product.

In one embodiment of the present disclosure, the chiral manganese dioxide nanoparticle is administered by intraperitoneal injection.

In one embodiment of the present disclosure, the dose of the chiral manganese dioxide nanoparticle is 40 mg/kg-60 mg/kg.

In one embodiment of the present disclosure, the chiral manganese dioxide is injected at a frequency of once every 1-3 days for 1-2 consecutive months.

Compared with the prior art, the above technical solutions of the present disclosure have the following advantages.

The present disclosure provides a chiral manganese dioxide nanoparticle, a preparation method therefor, and use thereof. The chiral manganese dioxide nanoparticle prepared according to the present disclosure has a strong circular dichroism signal in the visible light region, so that interference from signals in the small molecule ultraviolet region in an organism can be avoided.

The prepared chiral manganese dioxide nanoparticle of the present disclosure undergoes a redox reaction with ROS, and the resulting change in the circular dichroism signal intensity shows a good linear relationship with the concentration of ROS, so that the quantitative detection of ROS is achieved.

The prepared chiral manganese dioxide nanoparticle of the present disclosure can effectively eliminate ROS in aged skin, increase dermis thickness and collagen proportion, and reduce the oxidative stress level in skin tissue, and is beneficial to delaying skin aging.

### BRIEF DESCRIPTION OF THE DRAWINGS

In order to facilitate a clear understanding of contents of the present disclosure, the present disclosure will be further illustrated in detail below according to specific embodiments of the present disclosure in conjunction with the drawings. In the drawings:

FIG. 1 is a transmission electron microscopy image of the chiral manganese dioxide nanoparticles in Example 1 of the present disclosure;

FIG. 2 is a diagram showing the circular dichroism spectrum of the chiral manganese dioxide nanoparticles in Example 1 of the present disclosure;

FIG. 3 is a diagram showing the circular dichroism spectrum of the chiral manganese dioxide nanoparticles as a function of hydrogen peroxide concentration in Example 2 of the present disclosure;

FIG. 4 is a diagram showing the reaction mechanism of eliminating active oxygen by the chiral manganese dioxide nanoparticles in Example 3 of the present disclosure;

FIG. 5 is a diagram showing the improvement in the skin condition of aged mice by the chiral manganese dioxide nanoparticles in Example 4 of the present disclosure; and

FIG. 6 is a statistical diagram showing the reduction in the oxidative stress level and the improvement in the expression of oxidoreductase in skin tissues by chiral manganese dioxide in Example 5 of the present disclosure.

### DETAILED DESCRIPTION OF THE PREFERRED EMBODIMENTS

The present disclosure will be further described below with reference to the drawings and specific embodiments, such that those skilled in the art can better understand and implement the present disclosure. However, the embodiments should not be construed as limiting the present disclosure.

### Example 1. Method for Preparing Chiral Manganese Dioxide Nanoparticles

Synthesis of chiral MnO₂ nanoparticles using D-gluconic acid as the ligand: At room temperature, 50 mL of an ethylene glycol solution, 10 mL of a 100 mmol/L manganese chloride solution, and 20 mL of a 100 mmol/L D-gluconic acid solution were mixed homogeneously, and the mixture was stirred at room temperature for 10 min. Then, 20 mL of a 1 mmol/L sodium hydroxide solution was added, and the mixture was stirred at room temperature for another 6 h until the solution finally turned brownish red. The obtained product was added to absolute ethanol at a volume ratio of 1:3 to give a milky and turbid liquid, which was then centrifuged at a rotation speed of 8000 rpm/min for 10 min. The precipitate was chiral manganese dioxide nanoparticles, which were resuspended in ultrapure water for characterization and subsequent experiments.

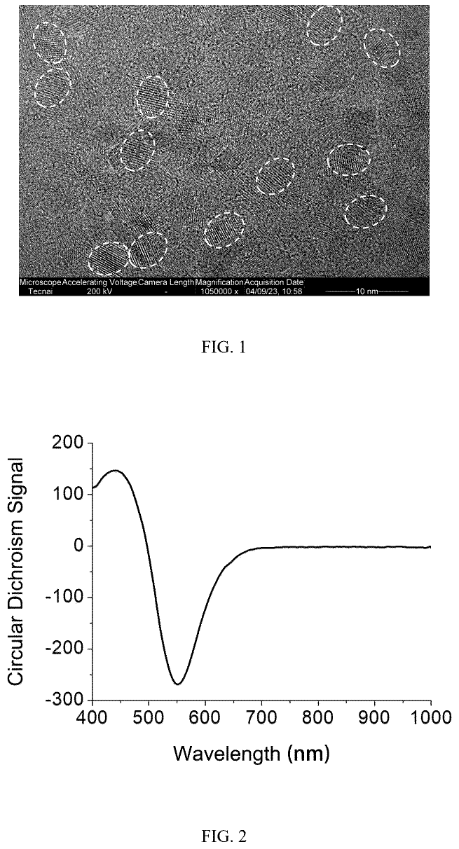

As can be seen from the transmission electron microscopy characterization in FIG. 1, the prepared nanoparticles have good chiral dispersibility and uniform particle size, with a diameter of 5 nm. Meanwhile, as can be seen from the circular dichroism spectrum in FIG. 2, the MnO₂ nanoparticles have relatively strong chiral signals, and the circular dichroism spectrum shows two significant peaks at 440 nm and 553 nm.

### Example 2. Method for Quantitative Detection of ROS by Chiral Manganese Dioxide Nanoparticles

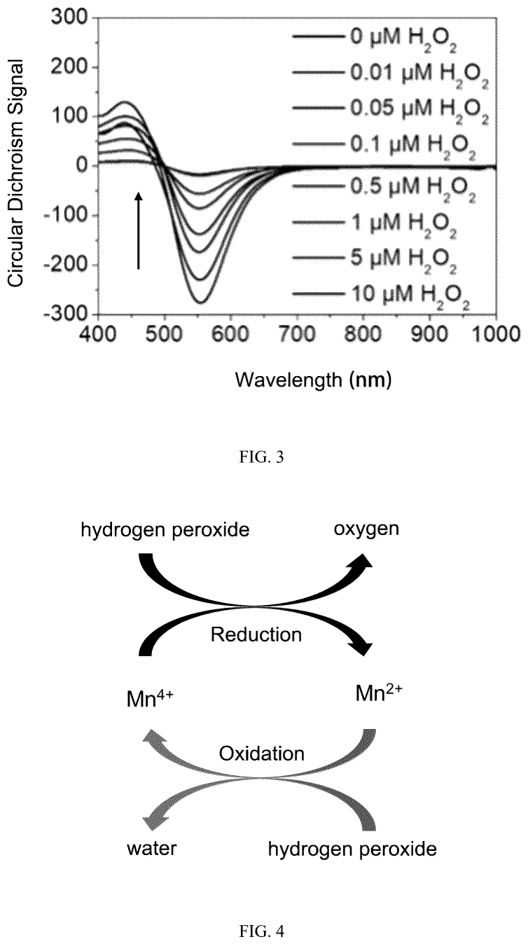

In the in vitro experiment, hydrogen peroxide solutions at different concentrations were added to chiral manganese dioxide nanoparticle solutions at the same concentration, and the mixtures were reacted for 30 s, followed by circular dichroism signal characterization. The results show a linear drop in circular dichroism signals with increasing concentration of the hydrogen peroxide solution (FIG. 3). The intensity of circular dichroism signals at 553 nm can be used to quantitatively detect the hydrogen peroxide concentration in the system.

### Example 3. Reaction Mechanism Between Chiral Manganese Dioxide Nanoparticles and ROS

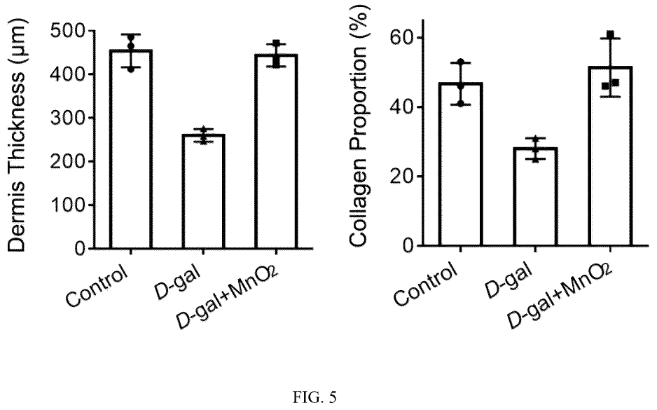

The valence of manganese in the solution before and after the reaction in Example 2 was tested by X-ray photoelectron spectroscopy. The divalent manganese and tetravalent manganese did not change significantly before and after the reaction, and the tetravalent manganese was reduced into divalent manganese and oxygen in the presence of hydrogen peroxide. In addition, the divalent manganese was further reacted with hydrogen peroxide and oxidized into tetravalent manganese and water (FIG. 4).

### Example 4. Improvement in Mouse Dermal Tissue Thickness and Collagen Proportion by Chiral Manganese Dioxide

Wild-type mice were randomly divided into three groups, with three mice in each group. The mice in one group were subcutaneously injected with 0.2 mL of normal saline as the negative control; the mice in another group were injected with 0.2 mL of D-galactose (200 mg/kg) once daily for 8 weeks to induce a skin aging model; the mice in the remaining group were injected with 0.2 mL of D-galactose (200 mg/kg) once daily for 8 weeks and then injected with 0.2 mL of chiral manganese dioxide nanoparticles at a dose of 50 mg/kg 6 h after daily injection of D-galactose from the fifth week. All mice were sacrificed after 8 weeks, and skin tissues were collected to measure the thickness and collagen proportion of the dermal tissues of the mice. The detection results in FIG. 5 show that mice with skin aging injected with the chiral manganese dioxide nanoparticles exhibited a significant increase in the dermis thickness and collagen proportion.

### Example 5. Reduction in Oxidative Stress Level and Improvement in Activity of Oxidoreductase in Skin Tissues by Chiral Manganese Dioxide

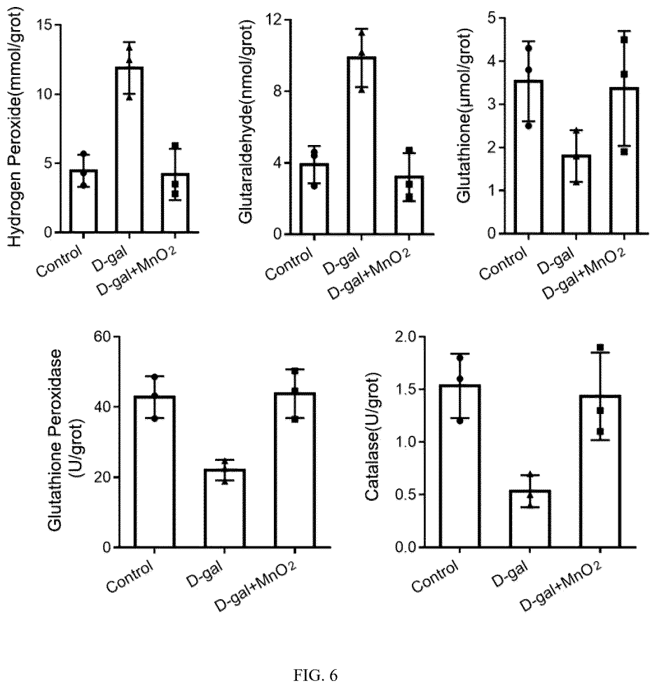

The skin tissue samples obtained in Example 4 were analyzed using assay kits to detect the oxidative stress level and the activity of antioxidase in the skin tissues. As can be seen from FIG. 6, the oxidative stress level was reduced and the antioxidase activity was increased in the skin tissues of the mice injected with the chiral manganese dioxide nanoparticles.

In this example, the hydrogen peroxide kit was purchased from Beyotime Biotechnology Co., Ltd., with Cat. No. S0038; the lipid oxidation kit was purchased from Beyotime Biotechnology Co., Ltd., with Cat. No. S0131S; the glutathione kit was purchased from Beyotime Biotechnology Co., Ltd., with Cat. No. S0052; the glutathione peroxidase kit was purchased from Beyotime Biotechnology Co., Ltd., with Cat. No. S0056; the catalase kit was purchased from Beyotime Biotechnology Co., Ltd., with Cat. No. S0051.

It is obvious that the above examples are merely illustrative for clear illustration and are not intended to limit the embodiments. Various changes and modifications can be made by those of ordinary skill in the art on the basis of the above description. It is unnecessary and impossible to exhaust all the embodiments herein. Obvious changes or modifications derived therefrom still fall within the protection scope of the present disclosure.

1. A method for preparing a chiral manganese dioxide nanoparticle, comprising steps of:
mixing a manganese source with chiral gluconic acid in a solvent, and adjusting the pH to alkalinity to give the chiral manganese dioxide nanoparticle.

2. The preparation method according to claim 1, wherein one or more of the following conditions are met:
(1) the chiral gluconic acid is D-gluconic acid or L-gluconic acid;
(2) the manganese source is selected from manganese chloride and/or manganese nitrate;
(3) the solvent is selected from the group consisting of ethylene glycol, ethanol, polyethylene glycol and any combination thereof.

3. The chiral manganese dioxide nanoparticle prepared by the method according to claim 1.

4. The chiral manganese dioxide nanoparticle according to claim 3, wherein one or two of the following conditions are met:
(a) the chiral manganese dioxide nanoparticle has a particle size of 3 nm-20 nm;
(b) the chiral manganese dioxide particle has a circular dichroism spectral signal at 400 nm-700 nm.

5. Use of the chiral manganese dioxide nanoparticle according to claim 3 in the detection of ROS.

6. The use according to claim 5, wherein the ROS is at a concentration of 0.01 μM-100 μM.

7. A composition, comprising the chiral manganese dioxide nanoparticle according to claim 3.

8. Use of the chiral manganese dioxide nanoparticle according to claim 3 in the preparation of an anti-aging skincare product.

9. The use according to claim 8, wherein the skincare product is a lotion, a cream, a facial mask, an essence, a skincare toner, a facial cleanser, or a gel.

10. Use of the chiral manganese dioxide nanoparticle according to claim 3 in the preparation of an anti-aging medicament or healthcare product.
